(19) United States
(12) Patent Application Publication
(10) Pub. No.: US 2026/0013965 A1
(43) Pub. Date: Jan. 15, 2026

(54) MEDICAL DEVICE PACKAGING CONTAINER

(71) Applicant: Corramedical, Inc., Lakeside, CA (US)

(72) Inventors: Alexander ARROW, Lakeside, CA (US); Wilfrido MOJICA, Amherst, NY (US)

(21) Appl. No.: 18/770,387

(22) Filed: Jul. 11, 2024

(51) Int. Cl. A61B 50/30

(52) U.S. Cl. CPC A61B 50/30

(57) ABSTRACT

A packaging container for a medical device is disclosed herein. In an embodiment, the packaging container includes an inner part and an outer part. The inner part has a rack surface with at least one cutout configured to hold one or more medical device both during shipping and upright during use. The outer part encloses the inner part and includes a tear line enabling a user to remove a portion thereof and expose the rack surface so that the user can use the medical device while held upright. In an embodiment, the inner part is formed by a single sheet of cardboard folded along a plurality of fold lines to form the rack surface, and the outer part is formed by a single sheet of cardboard folded along a plurality of fold lines to form the enclosure.

### BACKGROUND

### Technical Field

The present disclosure generally relates to a packaging container for a medical device. More specifically, the present disclosure relates to a packaging container that can be used to ship one or more of the medical devices and then also assist in the method of using the medical devices.

### Related Applications

The packaging of the present disclosure can be used in combination with the apparatuses and methods disclosed in U.S. patent application Ser. No. 18/468,416, filed Sep. 15, 2023, entitled “Methods and Systems for Recovering Assessable Analytes from Core Needle Biopsies,” U.S. patent application Ser. No. 18/514,870, filed Nov. 20, 2023, entitled “Apparatuses and Methods for Segregating Tissue Samples for Multiple Diagnostic Modalities,” and U.S. patent application Ser. No. 18/403,550, filed Jan. 3, 2024, entitled “Apparatuses and Methods for Segregating Tissue Samples for Multiple Diagnostic Modalities,” the entire contents of each of which are incorporated herein by reference.

### BACKGROUND INFORMATION

Solid tumor diagnostic procedures typically involve a tissue biopsy. Traditionally, a biopsy involves a substantial amount of tissue being surgically excised from a tumor or suspected affected tissue in a patient. The tissue, once removed from the patient's body, is processed and subsequently can be used for a number of different types of diagnostic tests.

In recent years, biopsy tools and techniques have advanced to be less invasive, with dramatically smaller tissue samples. Surgically-excised biopsies have largely been replaced by core needle biopsy (CNB) tools. Smaller biopsies are less traumatic for patients, quicker for the clinician to perform, and less expensive for the healthcare system in general. Hence, standard biopsy tissue size has declined significantly between the period before approximately 2010 and the years thereafter. The disadvantage of smaller biopsies is that they provide less tissue for pathologists to examine and analyze to render diagnostic opinions.

At the same time, diagnostic testing modalities have expanded to include an increased number of tests aimed at identifying molecular changes. The declining tissue biopsy size and the expanding quantity of testing required of the biopsied tissue has created an imbalance between tissue supply and demand. The result is that in some cases, clinicians make treatment decisions for patients with less diagnostic information than they would like. In other cases, patients are subjected to a second biopsy. The risk that a biopsy sample will have insufficient tissue to allow for the clinically-indicated tests to be performed is a big enough problem that it has several unofficial names, with “Tissue Exhaustion” being the most common. Tissue Exhaustion rates for core needle biopsies are reported in literature to be between 22-82% of all biopsies.

An imbalance therefore exists between the typical amount of tissue yielded from a CNB and the typical amount of tissue needed for testing. Healthcare quality is impacted by the shortfall in the quality and quantity of substrate available for molecular testing. This ultimately affects patient care, with many specimens received in the pathology laboratory not being available for molecular testing, resulting in these patients missing out on the improved treatment options associated with precision medicine (defined as using molecular testing to find a mutation to guide therapy).

It is unfortunate that standard tissue biopsy handling practices today result in some of the harvested cells being discarded along with medical waste. These cells come from the patient, unavoidably dislodged (referred to hereafter as D-cells) from the tissue due to the trauma associated with of the sharp edge of the CNB needle cutting through tissue and then pulling back into the metal CNB tube. These cells are not visible to the human eye. They contain valuable genetic information, but they are simply not noticed by the clinician or technician holding the CNB needle handle and placing the tissue into a standard formalin-containing cup-like container after harvesting then discarding the entire needle and any D-cells on it into a medical waste container. The current standard of care involves fixation of the tissue with formalin, in a process called formalin-fixation, paraffin embedding (FFPE), and which is known to create sub-optimal results when any tissue subjected to it is used as a substrate in molecular testing.

### SUMMARY

The present disclosure describes a new type of packaging for medical devices. More specifically, the present disclosure provides a new type of packaging for a new medical device that allows a clinician who performs a core needle biopsy to deposit the harvested tissue in such a way that it recovers cells that are dislodged (“D-cells”) on the needle from the solid tissue that is procured during the biopsy procedure or from the patient's bodily tissue surrounding the pathway taken by the needle. The packaging can also be used for other medical devices, for example, which similarly require upright components or liquids or tissue samples that are susceptible to spilling during shipping and/or a medical procedure.

As discussed in more detail below, the medical device and its packaging container disclosed herein enable a clinician who performs a core needle biopsy to deposit the tissue in such a way that it contains and preserves the microscopic accompanying portions (D-cells) of the biopsied tissue that would otherwise be inadvertently discarded. The medical device disclosed herein enables cells that are dislodged from tissue that is procured during a biopsy procedure or from the patient's bodily tissue surrounding the pathway taken by the needle to be kept and segregated from the tissue that will be sent for standard pathology laboratory processing. The packaging container disclosed herein secures this or other medical devices during shipping while also assisting with a medical procedure involving the medical device.

A first aspect of the present disclosure is to provide a packaging container for one or more medical device. The packaging container includes an inner part and an outer part. The inner part includes a rack surface having at least one cutout configured to hold the one or more medical device both during shipping and upright during use of the one or more medical device. The outer part encloses the inner part and includes a tear line enabling a user to remove a portion thereof and expose the rack surface of the inner part so that the user can use the one or more medical device while the one or more medical device is held upright by the inner part.

A second aspect of the present disclosure is to provide another packaging container for one or more medical device. The packaging container includes an inner part and an outer part. The inner part is formed by a first single sheet of cardboard folded along a plurality of fold lines to form at least a rack surface configured to hold the one or more medical device both during shipping and upright during use of the one or more medical device. The outer part is formed by a second single sheet of cardboard folded along a plurality of fold lines to form an enclosure for the inner part. The outer part includes an upper portion and a lower portion. The upper portion is configured to be separated from the lower portion to expose the inner part.

A third aspect of the present disclosure is to provide a medical package. The medical package includes one or more medical device and a packaging container. The packaging container contains the one or more medical device therein. The packaging container has at least one cutout configured to hold the one or more medical device both during shipping and upright during use of the one or more medical device.

A fourth aspect of the present disclosure is to a method of using a packaging container for one or more medical device before, during, and/or after a medical procedure such as a core needle biopsy.

Other objects, features, aspects and advantages of the apparatuses and methods disclosed herein will become apparent to those skilled in the art from the following detailed description, which, taken in conjunction with the annexed drawings, discloses exemplary embodiments of the disclosed apparatuses and methods.

### BRIEF DESCRIPTION OF THE DRAWINGS

Referring now to the attached drawings which form a part of this original disclosure:

FIG. 1 illustrates a perspective view of an example embodiment of a packaging container in accordance with the present disclosure;

FIG. 2 illustrates an outer part of the packaging container of FIG. 1 before the outer part is fully constructed and combined with the inner part shown in FIG. 3;

FIG. 3 illustrates the inner part of the packaging container of FIG. 1 before the inner part is constructed and combined with the outer part shown in FIG. 2;

FIG. 4 illustrates a top perspective view of the inner part of the packaging container of FIG. 1 with the inner part constructed;

FIG. 5 illustrates a bottom perspective view of the inner part of the packaging container of FIG. 1 with the inner part constructed;

FIG. 6 illustrates a top perspective view of the packaging container of FIG. 1 after a portion thereof has been removed as described herein;

FIG. 7 illustrates another top perspective view of the packaging container of FIG. 1 after a portion thereof has been removed as described herein;

FIG. 8 illustrates another top perspective view of the packaging container of FIG. 1 including a plurality of individually packaged medical devices in accordance with the present disclosure;

FIG. 9 illustrates a side plan view an example embodiment of a medical device for use with the packaging container of FIG. 1;

FIG. 10 illustrates a cross-sectional view of the medical device of FIG. 1 taken across lines X-X in FIG. 9;

FIG. 11 illustrates an exploded view of the cross-section for the medical device shown in FIG. 10;

FIG. 12 illustrates an example embodiment of a method of using the medical device of FIGS. 9 to 11 with the packaging container of FIGS. 1 to 8 in accordance with the present disclosure;

FIG. 13 illustrates a top perspective view of the medical device of FIGS. 9 to 11 being used with the packaging container of FIGS. 1 to 8 in accordance with the present disclosure;

FIG. 14 illustrates a top perspective view of the medical device of FIGS. 9 to 11 being used with the packaging container of FIGS. 1 to 8 in accordance with the present disclosure;

FIG. 15 illustrates a top perspective view of the medical device of FIGS. 9 to 11 being used with the packaging container of FIGS. 1 to 8 in accordance with the present disclosure;

FIG. 16 illustrates a top perspective view of the medical device of FIGS. 9 to 11 being used with the packaging container of FIGS. 1 to 8 in accordance with the present disclosure;

FIG. 17 illustrates a top perspective view of another example embodiment of a packaging container in accordance with the present disclosure;

FIG. 18 illustrates another top perspective view of the packaging container of FIG. 17;

FIG. 19 illustrates a top perspective view of the inner part of the packaging container of FIG. 17 with the inner part constructed;

FIG. 20 illustrates a bottom perspective view of the inner part of the packaging container of FIG. 17 with the inner part constructed; and

FIG. 21 illustrates a top perspective view of the constructed parts of the packaging container of FIG. 1 and the packaging container of FIG. 17.

### DETAILED DESCRIPTION OF EMBODIMENTS

Selected embodiments will now be explained with reference to the drawings. It will be apparent to those skilled in the art from this disclosure that the following descriptions of the embodiments are provided for illustration only and not for the purpose of limiting the invention as defined by the appended claims and their equivalents.

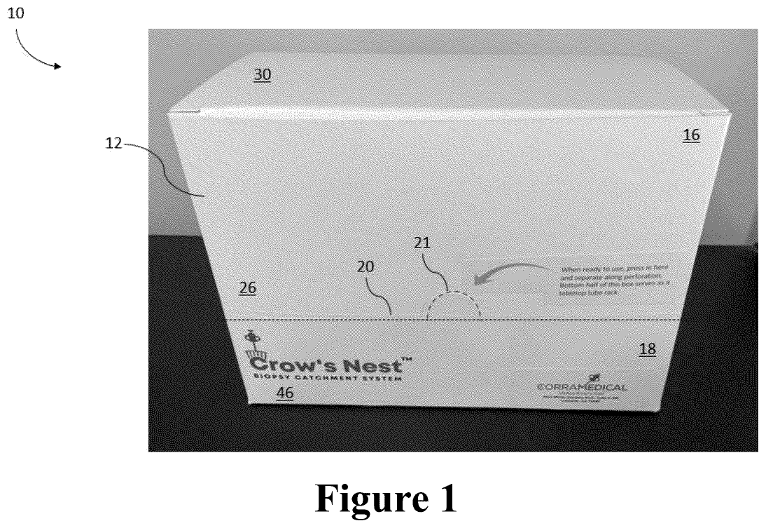

FIGS. 1 to 8 illustrate an example embodiment of a packaging container 10 in accordance with the present disclosure. The packaging container 10 can be used to ship one or more medical device 100 and then also to assist in a method of using the one or more medical device 100, as described in more detail below. Thus, in an embodiment, a medical package includes the packaging container 10 and one or more medical device 100.

In the illustrated embodiment, the packaging container 10 includes an outer part 12 and an inner part 14. In use, the inner part 14 is configured to hold one or more medical device 100 both during shipping and upright during use of the medical device 100, while the outer part 12 is configured to form an enclosure for the inner part 14 which can be separated to expose the inner part 14 during use of the medical device 100. In the illustrated embodiment, the inner part 14 is formed by a first single sheet of cardboard folded along a plurality of fold lines to form at least a rack surface configured to hold one or more medical device 100 both during shipping and upright during use of the medical device 100, and the outer part 12 is formed by a second single sheet of cardboard folded along a plurality of fold lines to form an enclosure for the inner part 14.

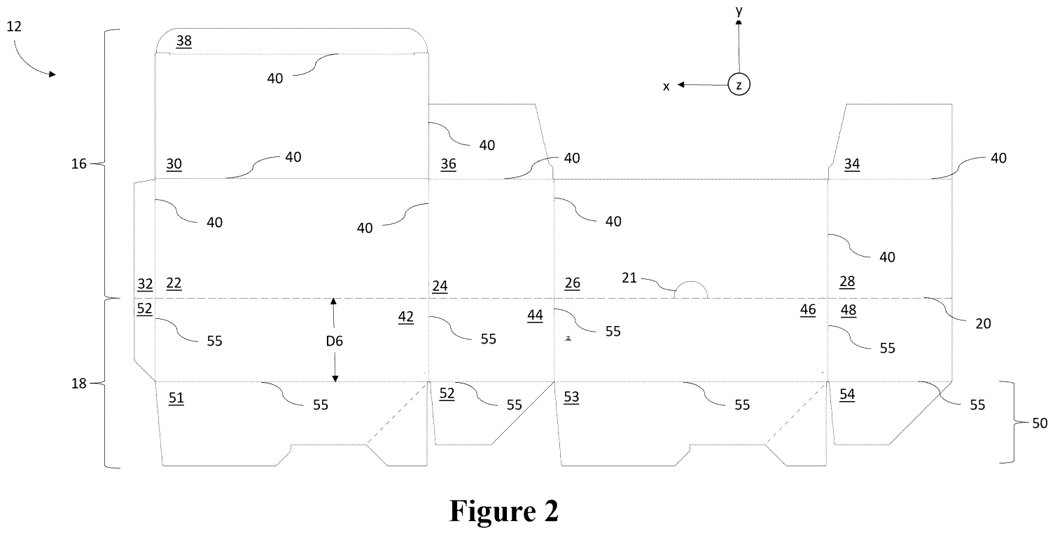

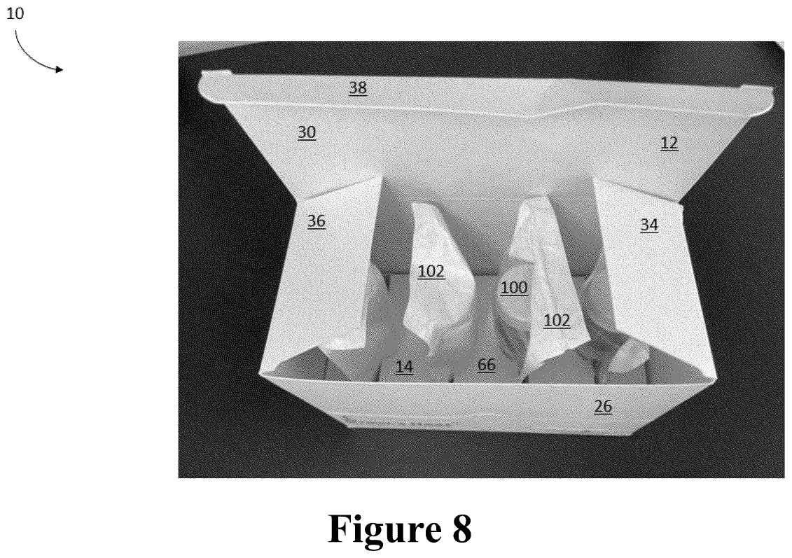

FIG. 2 shows the outer part 12 before the outer part 12 is fully constructed and combined with the inner part 14. In the illustrated embodiment, the outer part 12 is a single flat sheet of cardboard that is cut around the outer perimeter shown in FIG. 2 before being folded and and/or adhered to itself to appear as shown in FIGS. 1 and 8. Those of ordinary skill in the art will recognize from this disclosure that the outer part 12 can be formed of different types or thicknesses of cardboard, or can be formed of other materials such as a thin plastic.

In the illustrated embodiment shown in FIG. 2, the outer part 12 includes an upper portion 16 and a lower portion 18. The outer part 12 also includes a tear line 20 that separates the upper portion 16 and the lower portion 18. The upper portion 16 of the outer part 12 includes a first side surface 22, a second side surface 24, a third side surface 26, a fourth side surface 28, a top surface 30, and one or more free ends 32, 34, 36, 38, all of which are connected as shown by fold lines 40. The lower portion 18 of the outer part 12 includes a first side surface 42, a second side surface 44, a third side surface 46, a fourth side surface 48, and a bottom surface 50 formed by one or more free ends 51, 52, 53, 54, all of which are connected as shown by fold lines 55. The upper portion 16 is configured to be separated from the lower portion 18 using the tear line 20 so that the inner part 14 is exposed to enable a user to use one or more medical device 100 while held upright by the inner part 14. The lower portion 18 contains the inner part 14 when the upper portion 16 is removed using the tear line 20.

When the outer part 12 is folded across the fold lines 40, 55 and attached to itself using one or more of the free ends 32, 34, 36, 38, 52, 53, 54, the outer part 12 forms the rectangular box shape seen in FIGS. 1 and 8. In the illustrated embodiment, from the view seen in FIG. 2, the parts of the upper portion 16 are all folded across the fold lines 40, 55 in the same direction (into the paper or in the −z direction shown). In the illustrated embodiment, the free end 32 is adhered to the fourth side surface 28, the free end 52 is adhered to the fourth side surface 48, and/or one or more of the free ends 51, 52, 53, 54 are adhered to each other. The parts can be adhered, for example, using a liquid adherence such as glue, a physical adherence such as tape, or another type of adherence.

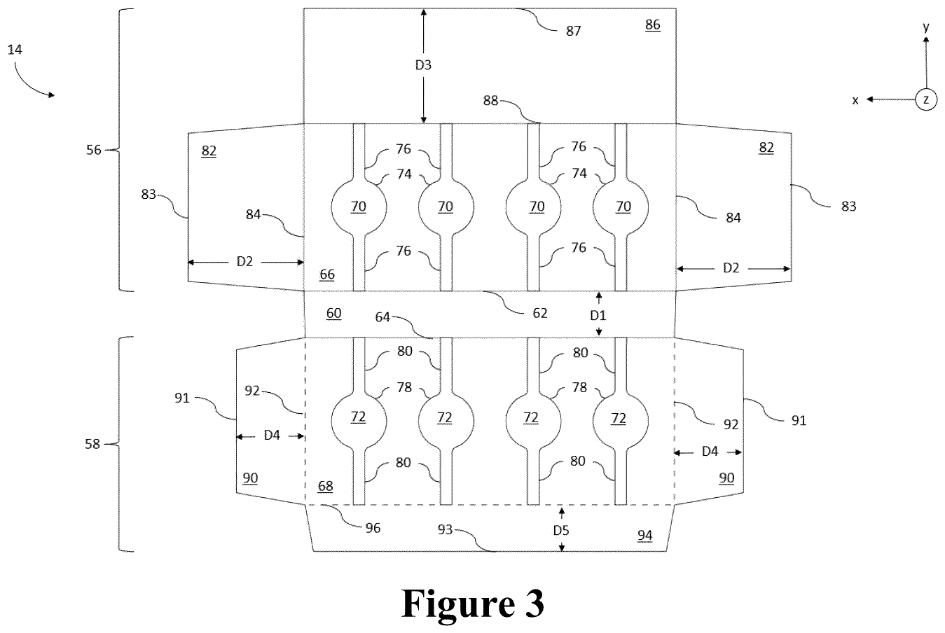

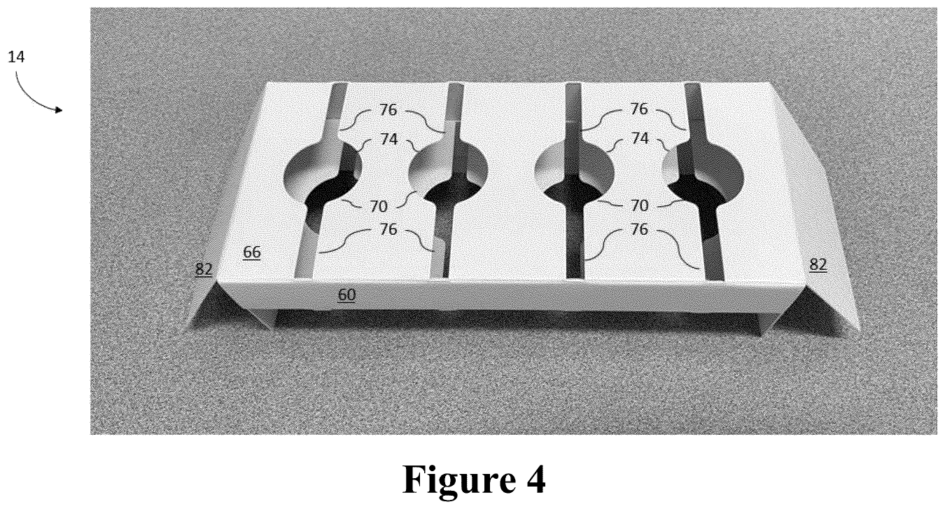

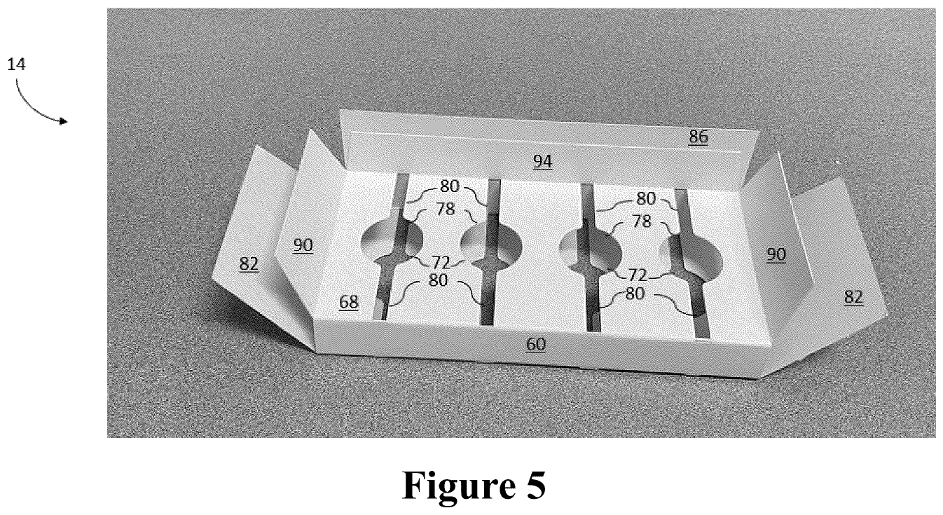

FIG. 3 shows the inner part 14 before the inner part 14 is fully constructed and combined with the outer part 12. In the illustrated embodiment, the inner part 14 is a single flat sheet of cardboard that is cut around the outer perimeter shown in FIG. 3 before being folded and and/or adhered to itself to appear as shown in FIGS. 4 and 5. Those of ordinary skill in the art will recognize from this disclosure that the inner part 14 can be formed of different types or thicknesses of cardboard, or can be formed of other materials such as plastic. In an embodiment, both the outer part 12 and the inner part 14 can be each part of a same single sheet of cardboard.

In the illustrated embodiment, the inner part 14 includes an upper portion 56 and a lower portion 58. The inner part 14 also includes an intermediate portion 60 located between the upper portion 56 and the lower portion 58. The upper portion 56 is connected to the intermediate portion 60 by a fold line 62, and the lower portion 58 is connected to the intermediate portion 60 by a fold line 64.

As seen in FIG. 3, the inner part 14 includes an upper rack surface 66 and a lower rack surface 68. In the illustrated embodiment, the upper portion 56 includes the upper rack surface 66, and the lower portion 58 includes the lower rack surface 68. More specifically, the upper portion 56 includes the upper rack surface 66 connected to the intermediate portion 60 by the fold line 62, and the lower portion 58 includes the lower rack surface 68 connected to the intermediate portion 60 by the fold line 64.

In the illustrated embodiment, the upper rack surface 66 includes one or more upper cutouts 70. The upper cutouts 70 are configured to hold one or more medical device 100 upright during use of the medical device 100. More specifically, the upper cutouts 70 are configured to hold one or more medical device 100 both during shipping and upright during use of the medical device 100. Similarly, in the illustrated embodiment, the lower rack surface 68 includes one or more lower cutouts 72. The lower cutouts 72 are also configured to hold one or more medical device 100 upright during use of the medical device 100. More specifically, the lower cutouts 72 are configured to hold one or more medical device 100 both during shipping and upright during use of the medical device 100. At least one of the upper or lower cutouts 70, 72 can be formed as an aperture in at least one of the upper or lower rack surfaces 66, 68. In the embodiment shown in FIGS. 4 to 7, each of the lower cutouts 72 align with an upper cutout 70 in the vertical direction.

In the illustrated embodiment shown in FIG. 3, the upper cutouts 70 are apertures in the upper rack surface 66, and the lower cutouts 72 are apertures in the lower rack surface 68. As seen in FIG. 3, the upper cutouts 70 each include a center portion 74 and at least one side portion 76. More specifically, the upper cutouts 70 each include a center portion 74 and two side portions 76. The side portions 76 form an aperture in combination with the center portion 74. The side portions 76 extend outwardly from the center portion 74 in a first direction (y-direction in FIG. 3) and are smaller than the center portion 74 in a second direction perpendicular to the first direction (x direction in FIG. 3). Likewise, the lower cutouts 72 each include a center portion 78 and at least one side portion 80. More specifically, the lower cutouts 72 each include a center portion 78 and two side portions 80. The side portions 80 form an aperture in combination with the center portion 78. The side portions 80 extend outwardly from the center portion 78 in a first direction (y-direction in FIG. 3) and are smaller than the center portion 78 in a second direction perpendicular to the first direction (x direction in FIG. 3). In the illustrated embodiment, the center portions 74, 78 are circular, and the side portions 76, 80 are generally rectangular and extend from opposite sides of the center portions 74, 78 to opposing fold lines 62, 88 or opposing fold lines 64, 94, respectively. In the illustrated embodiment, the upper cutouts 70 and the lower cutouts 72 have the same size, shape and dimensions. Those of ordinary skill in the art will recognize from this disclosure that the upper cutouts 70 and the lower cutouts 72 can be made in different sizes, shapes or dimensions and/or be placed at different locations.

In the illustrated embodiment, the upper portion 56 includes upper side surfaces 82 attached to the upper rack surface 66 by fold lines 84. The upper portion 56 also includes an upper lateral surface 86 attached to the upper rack surface 66 by a fold line 88. Similarly, the lower portion 58 includes lower side surfaces 90 attached to the lower rack surface 68 by fold lines 92. The lower portion 58 also includes a lower lateral surface 94 attached to the lower rack surface 68 by a fold line 96.

As seen in FIGS. 4 and 5, the inner part 14 is constructed by folding the lower portion 58 underneath the upper portion 56 using the fold lines 62 and 64, such that the upper rack surface 66 and the lower rack surface 68 are generally parallel and generally perpendicular to the surface of the intermediate portion 60. From the view of FIG. 3, the upper side surfaces 82 and upper lateral surface 86 are folded along the respective fold lines 84, 88 in the opposite direction that the lower side surfaces 90 and lower lateral surface 94 are folded along the respective fold lines 92, 96 (e.g., the −z vs +2 direction in FIG. 3). The solid and dashed fold lines in FIG. 3 identify the lines that fold in the opposite directions. That is, the broken lines in FIG. 3 identify the fold lines 92, 96 that fold in the opposite direction of the other fold lines 62, 64, 84, 88 identified with solid lines.

When the inner part 14 is constructed, each upper side surface 82 is placed against and/or parallel to a respective lower side surface 90, and the upper lateral surface 86 is placed against and/or parallel to the lower lateral surface 94. With this construction, the upper rack surface and the lower rack surface 68 are generally horizontal and generally parallel with each other, and the upper side surfaces 82, the upper lateral surface 86, the lower side surfaces 90 and the lower lateral surface 94 are generally vertical. In an embodiment, the parts of the inner part 14 are held in place by the outer part 16 without separate adherence needed. In an alternative embodiment, one or both of the upper side surfaces 82 can be adhered to respective a lower side surface 90, and/or the upper lateral surface 86 can be adhered to the lower lateral surface 94. The parts can be adhered, for example, using a liquid adherence such as glue, a physical adherence such as tape, or another type of adherence.

As seen in FIG. 3, the upper rack surface 66 and the lower rack surface 68 have generally the same dimensions in the x and y directions. The intermediate portion 60 has the same dimension as the upper rack surface 66 and the lower rack surface 68 in the x direction, but extends a different distance D1 in the y direction. The distance D1 defines the vertical spacing between the upper rack surface 66 and the lower rack surface 68 when the inner part 14 is constructed. The upper side surfaces 82 extend away from the fold lines 84 connecting to the upper rack surface 66 by a distance D2 in the x direction, and the upper lateral surface 86 extends away from the fold line 88 connecting to the upper rack surface 66 by a distance D3 in the y direction. The lower side surfaces 90 extend away from the fold lines 92 connecting to the lower rack surface 68 by a distance D4 in the x direction, and the lower lateral surface 94 extends away from the fold line 96 connecting to the lower rack surface 68 by a distance D5 in the y direction. In an embodiment, the distance D2 is greater than the distance D4, and the distance D3 is greater than the distance D5, so that when the lower rack surface 68 is folded underneath the upper rack surface 66, the respective outer edges 83, 91 of the upper side surfaces 82 and the lower side surfaces 90 generally align, and/or so that the respective outer edges 87, 93 of the upper lateral surface 86 and the lower lateral surface 94 generally align. In an embodiment, the distance D2 is approximately equal to the sum of the distance D1 and the distance D4. In another embodiment, the distance D3 is approximately equal to the sum of the distance D1 and the distance D5.

Referring again to FIG. 1, the packaging container 10 is shown with the outer part 12 and the inner part 14 constructed and the inner part 14 located inside the outer part 12. The side surface 26 includes a punch out section 21 (here, a half-circle) located adjacent to the tear line 20, such that a user can place a finger in the punch out section 21 to grip and tear the upper portion 16 off of the lower portion 18 along the tear line 20.

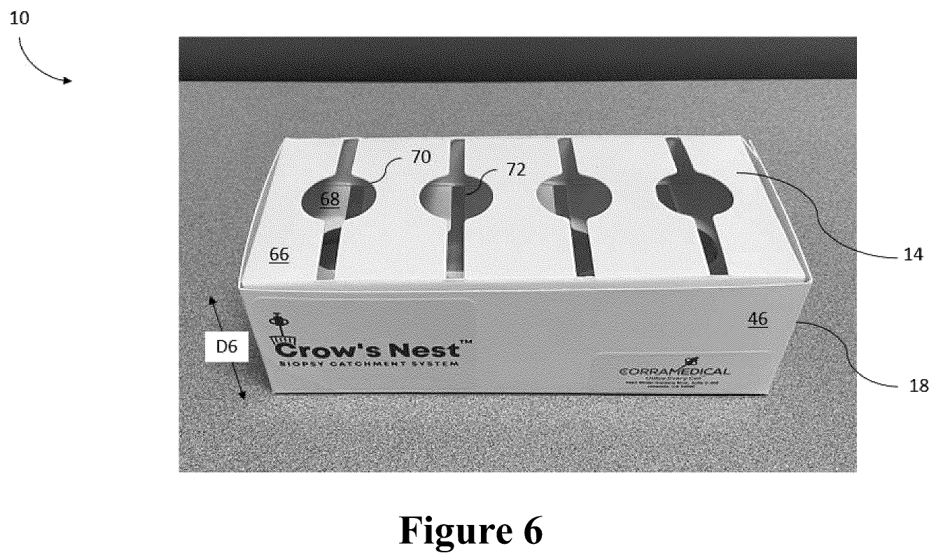

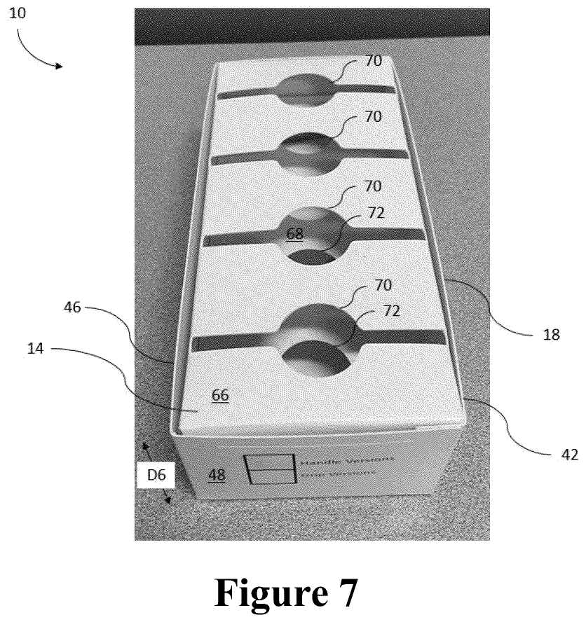

FIGS. 6 and 7 show the remaining packaging container 10 after the upper portion 16 is torn off of the lower portion 18 along the tear line 20. As illustrated, this exposes the inner part 14 so that the inner part can be used as a rack while using one or more medical device 100 packaged therein. As seen in FIGS. 6 and 7, the height of the upper rack surface 66 when the inner portion 14 is constructed is generally the same as the height D6 of the lower portion 18 with the upper portion 16 removed (e.g., the height D6 of the first side surface 42, the second side surface 44, the third side surface 46 and the fourth side surface 48 shown in FIG. 2).

FIG. 8 shows the fully constructed packaging container 10 with a plurality of medical devices 100 located therein. Each of the medical devices 100 is located within a respective upper cutout 70 and corresponding lower cutout 72. The packaging container 10 shown in FIG. 8, with the top surface 30 closed, is how the medical devices 100 are shipped, for example, to hospitals or clinical labs. In the illustrated embodiment, the medical devices 100 are also individually scaled in individual device packaging 102. As seen in FIG. 8, the medical devices 100 that are each individually sealed in individual device packaging 102 are each positioned within a respective cutout 70, 72. The center portions 74, 78 can be considered device portions, such that the medical devices 100 that are each individually scaled in individual device packaging 102 fit into respective cutouts 70, 72 with the medical device 100 located in a device portion (e.g., center portions 74, 78 of the cutouts 70, 72) and with the individual device packaging 102 located in the side portions 76, 80 of the cutouts 70, 72.

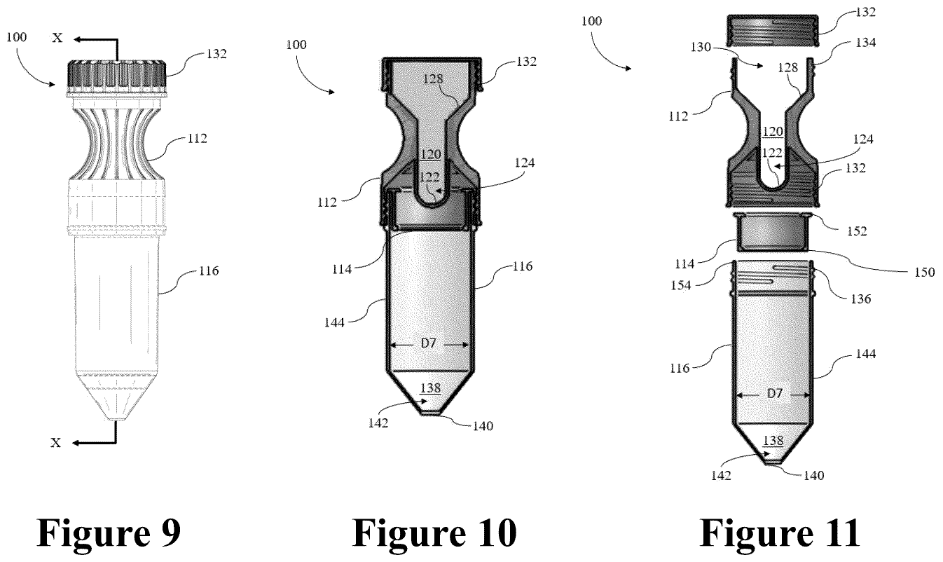

FIGS. 9 to 11 illustrate an example embodiment of a medical device 100 in accordance with the present disclosure. In the illustrated embodiment, the medical device 100 separates a tissue sample from dislodged cells (D-cells) obtained during a biopsy. The packaging container 10 disclosed herein is particularly advantageous for enabling use of the medical devices 100 shipped therein.

In the illustrated embodiment, the medical device 100 includes a buffer container 112 and a sample collection container 116. The buffer container 112 is removeable from the sample collection container 116 while the sample collection container 116 is help upright within at least one cutout 70, 72. More specifically, the medical device 100 includes a buffer container 112, a basket sieve 114 and a sample collection container 116, which are three separable elements that can be attached within the packaging container 10 and/or individual device packaging 102 and are then separated when used in combination with the packaging container 10. FIGS. 9 and 10 illustrate the medical device 100 with these parts attached together, while FIG. 11 illustrates these parts detached from each other. In the illustrated embodiment, the medical device 100 is used to separate solid tissue and D-cells taken in a biopsy using a core needle.

The buffer container 112 includes a buffer chamber 120 for storing or receiving buffer solution. The bottom edge 122 of the buffer chamber 120 is scaled and watertight so that the inner space 124 of buffer chamber 120 retains the buffer solution. In an embodiment, the buffer chamber 120 is pre-filled with the buffer solution within the inner space 124 when shipped within the packaging container 10. In an embodiment, the buffer solution is a sterile phosphate-buffered saline (PBS) buffer solution. In an embodiment, the buffer chamber 120 includes between 1 and 2 mL of buffer solution. In an embodiment, the buffer chamber 120 is pre-filled with approximately 1-2 ml of buffer solution. While PBS is the most likely choice of buffer, any similar buffer solution, such as Buffer Roswell Park Memorial Institute (“RPMI 1640 Media”) Buffer Solution, would serve the same purpose.

In the illustrated embodiment, the buffer container 112 includes a funnel to assist a user in depositing a biopsy sample from a core needle into the buffer solution within the buffer chamber 120 while the medical device is held upright by the packaging container 10. More specifically, the buffer container 112 includes a funnel portion 128 leading into the buffer chamber 120. The funnel portion 128 flares outwardly from bottom to top, while the buffer chamber 120 has a generally cylindrical shape. The funnel portion 128 is configured to guide solid tissue and the D-cells from a biopsy into the buffer chamber 120 when ejected from a core needle, as discussed in more detail below.

In the illustrated embodiment, the buffer container 112 includes a top opening 130 and a lid 132. The lid 132 attaches at or near the top of the funnel portion 124 to cover the top opening 130 and enclose the inner space 124 so that the buffer solution does not spill if the medical device 100 is inverted during shipping within the packaging container 10. In the illustrated embodiment, the lid 132 is configured to attach to both the buffer container 112 and the sample collection container 116, so that a user can remove the lid 132 from the buffer container 112 when beginning use of the medical device 100 and then later place the lid 132 on the sample collection container 116 to seal its contents. In an embodiment, the lid 132 is configured to attach to the buffer container 112 by threads 134 on the exterior of the buffer container 112, and to attach to the sample collection container 116 by the same or similar sized threads 136 on the exterior of the sample collection container 116.

The sample collection container 116 includes a reagent chamber 138 for storing or receiving a reagent. The bottom edge 140 of the sample collection container 116 is sealed and watertight so that the reagent chamber 138 retains the reagent. In an embodiment, the reagent chamber 138 is pre-filled with the reagent within an inner space 142. In an embodiment, the reagent is a solution for lysing cells and preserving nucleic acids that is approximately 2× the normal concentration of an off-the-shelf cell lysing reagent. In an embodiment, the reagent is Zymo DNA/RNA Shield™ reagent, or an equivalent for lysing cells and preserving nucleic acids which is 2× the normal concentration defined by and provided by Zymo. In an embodiment, the reagent chamber 138 includes between 1 and 2 mL of reagent. In an embodiment, the reagent chamber 138 is pre-filled with 1-2 ml of double-concentration cell lysis/nucleic acid stabilization reagent. In an embodiment, the reagent chamber 138 includes a first amount of reagent, and the buffer chamber 120 includes a second amount of buffer solution that is approximately equal in volume to the first amount of reagent. In the illustrated embodiment, the outer surface 144 of the sample collection container 116 has a diameter D7 that is approximately equal to or slightly smaller than the diameter of the center portions 74, 78 of the cutouts 70, 72 in the packaging container 10 so that the sample collection container 116 can be placed into and held in place within the cutouts 70, 72 during shipping and/or use of the medical device 100.

The basket sieve 114 includes a sieve surface 150 configured to pass the D-cells from a biopsy but not solid tissue from the biopsy. In an embodiment, the pore size of the sieve surface 150 is approximately 40-100 microns in diameter per pore (the pore aperture size) to allow D-cells to fall through. In the illustrated embodiment, the sieve surface 150 is the lower surface of the basket sieve 114. In an embodiment, the basket sieve 114 also includes a lip 152 sized to rest on the rim 154 of the sample collection container 116 so that the basket sieve 114 is suspended within the sample collection container 116 above the reagent chamber 138 and below the buffer chamber 120 when the medical device 100 is assembled as shown in FIGS. 9 and 10.

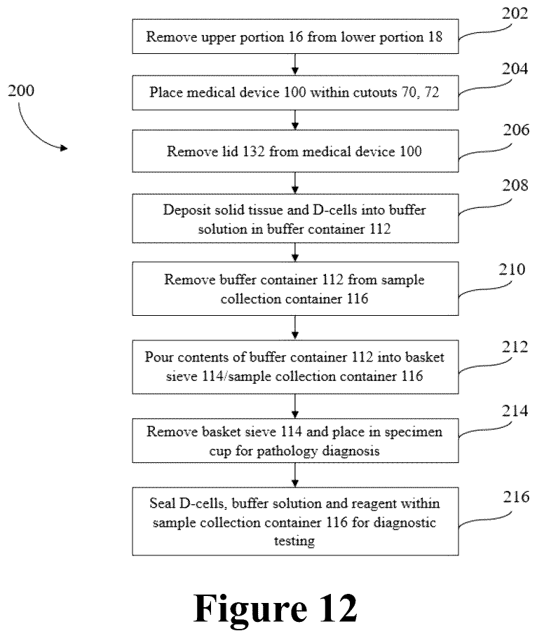

FIG. 12 illustrates an example embodiment of a method 200 of using the medical device 100 with the packaging container 10 in accordance with the present disclosure. In an embodiment, the method 200 is a method of recovering solid tissue and D-cells from a biopsy using the medical device 100 in combination with the packaging container 10. Those of ordinary skill in the art will recognize from this disclosure that certain steps of the method 200 can be added, removed or altered without departing from the spirit and scope of the present disclosure. Those of ordinary skill in the art will also recognize from this disclosure that certain steps of the method 200 can be used with other medical devices besides the medical device 100 disclosed herein.

Initially, in the illustrated embodiment, either before or during the method 200, a user (e.g., an interventional radiologist or other clinical user) uses a core needle CN to harvest an image guided biopsy from a patient with a suspicious mass lesion inside his or her body (e.g., liver, lung, kidney, etc). The core needle CN removes both solid tissue and dislodged cells (D-cells) from the patient.

At step 202, a user (e.g., the interventional radiologist or other clinical user), removes the upper portion 16 of the packaging container 10 from the lower portion 18 of the packaging container 10. In the illustrated embodiment, the user removes the upper portion 16 from the lower portion 18 by placing a finger in the punch out section 21 and separating the upper portion 16 from the lower portion 18 along the tear line 20. The user is then left with the lower portion 18 having the inner part 14 therein, as shown for example by FIGS. 6, 7 and 13 to 16.

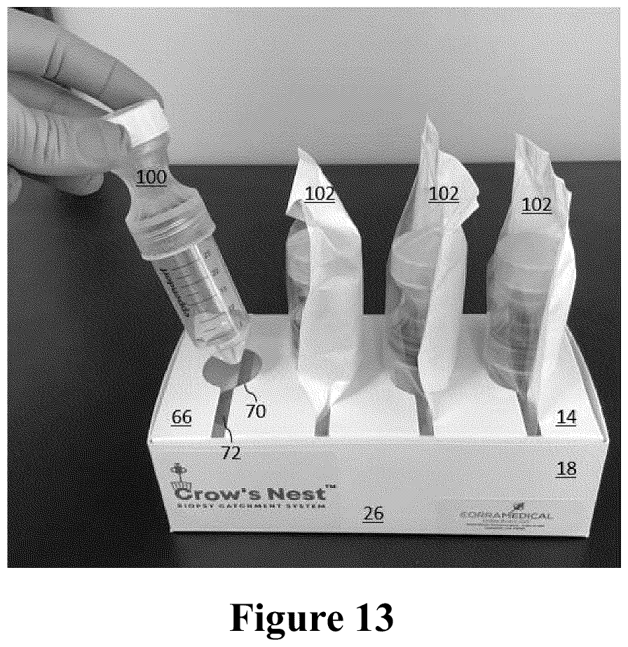

At step 204, the user removes one of the medical devices 100 from its individual device packaging 102 and places the medical device 100 within one or more cutouts 70, 72 so that the medical device 100 is held by the inner part 14 with its longitudinal axis (the vertical axis in FIGS. 9-11) in a generally vertical position. More specifically, the user places the medical device 100 within one or more device or center portions 74, 78 of the cutouts 70, 72 in the packaging container 10. FIG. 13 illustrates an example embodiment of step 204.

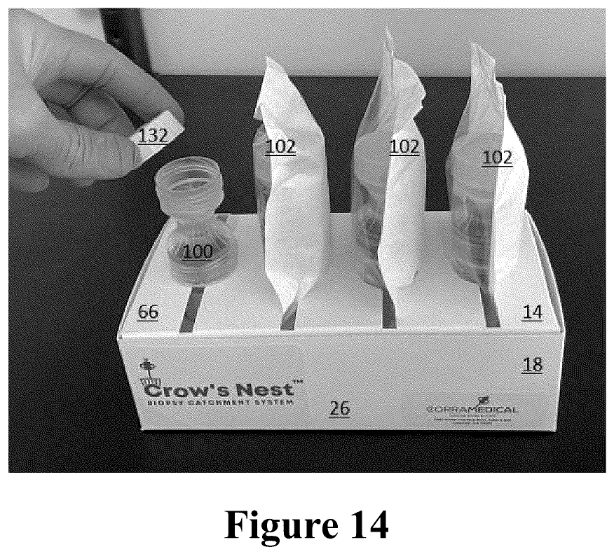

At step 206, the user removes the lid 132 from the medical device 100 but leaves the rest of the medical device 100 standing within the cutouts 70, 72. More specifically, the user removes the lid 132 from the buffer container 112 to open the top opening 130 of the buffer container 112. The lid 132 can be set aside and later reused to seal the sample collection container 116 at step 216. FIG. 14 illustrates an example embodiment of step 206.

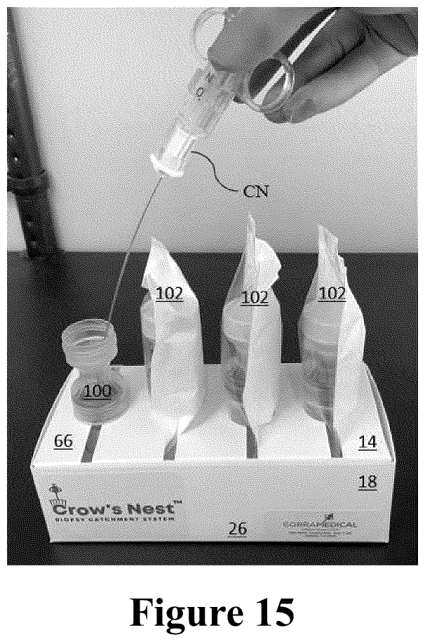

At step 208, the user deposits the contents of a core needle CN including the solid tissue and the D-cells into the buffer solution within the buffer chamber 120. The user may swirl the CNB needle tip to deposit the harvested tissue sample by moving the CNB needle tip in a roughly circular fashion. The funnel portion 128 makes it easy for the user to spatially coordinate the CNB needle tip and the target area and provides a convenient way to for the user to place the tip of the needle (and therefore the tissue sample) near or into the buffer solution. That is, the packaging 10 holding the medical device 100 upright, in combination with the larger funnel portion 128, makes the buffer chamber 120 easier to hit since the user's hand is approximately eight (8) inches away from the needle tip (holding the handle; not the tip), and the other hand is holding the buffer container 112 and/or the packaging container 10 or otherwise unavailable and generally not used to guide the tip of the needle. FIG. 15 illustrates an example embodiment of step 208.

At step 210, the user removes the buffer container 112 from the sample container 116. The user can remove the buffer container 112 from the sample collection container 116, for example, by detaching (e.g., unscrewing) the buffer container 112 from the sample collection container 116 or another portion of the medical device 100. The sample collection container 116 can remain in or be placed back into the cutouts 70, 72 once the buffer container 112 has been detached. The basket sieve 114 remains within the sample collection container 116 at this point.

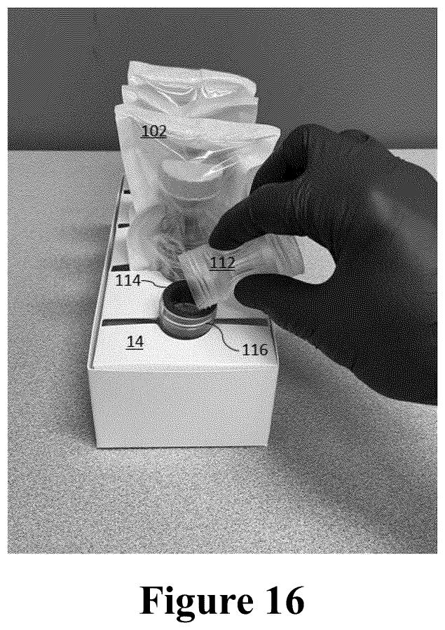

At step 212, the user pours the contents of the buffer container 112 (now including the solid tissue and the D-cells deposited from the core needle CN, as well as the buffer solution from the buffer chamber 120) into the basket sieve 114 that is located within the sample collection container 116 held within the cutouts 70, 72 of the packaging container 10. The solid tissue is captured by the sieve surface 150 and remains within the basket sieve 114, while the buffer solution and the D-cells fall through the sieve surface 150 and mix with the reagent in the reagent chamber 138 of the sample collection container 116. Since the reagent is 2× concentrated, the additional volume of buffer solution and dislodged cells restores the reagent concentration to normal. The user can then discard the buffer container 112. The sample collection container 116 can remain held within the cutouts 70, 72. FIG. 16 illustrates an example embodiment of step 204.

At step 214, the user removes the basket sieve 114 containing the solid tissue from the sample collection container 116 now containing the buffer solution, reagent and D-cells. In an embodiment, the user the places the basket sieve 114 containing the solid tissue into a specimen cup. The specimen cup can contain formalin. The specimen cup is sealed and sent to a tissue pathology lab. Thereafter the tissue core can undergo traditional tissue processing, for example, to create a glass slide image for the pathologist to make a diagnosis. The sample collection container 116 can remain in or be placed back into the cutouts 70, 72 once the basket sieve 114 has been detached.

At step 216, the user can remove the sample collection container 116 from the cutouts 70, 72 and seal the sample collection container 116 including the D-cells, the buffer solution and the reagent for diagnostic testing. In an embodiment, the user can seal the sample collection container 116 by attaching the lid 132 that was previously removed from the buffer container 112. The user can then send the sealed sample collection container 116 and its contents to a molecular lab for molecular diagnostic testing. At this point the buffer container 112 and the basket sieve 114 have been removed and the sealed sample collection container 16 includes the mixed buffer solution, reagent and D-cells.

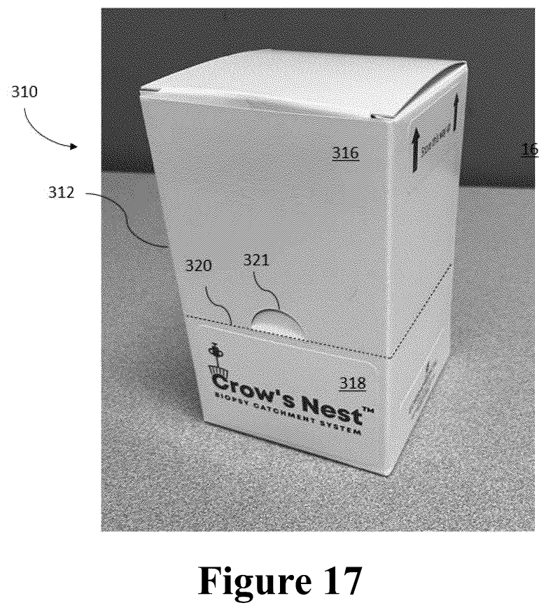

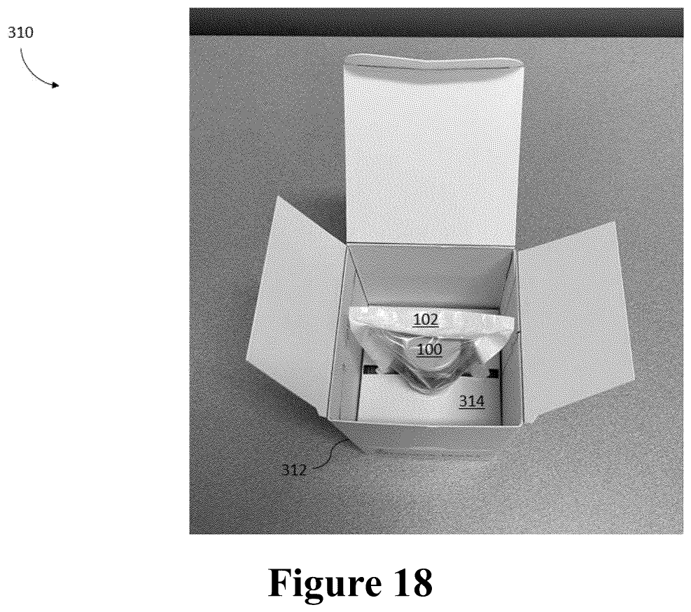

FIGS. 17 to 20 illustrate another example embodiment of a packaging container 310 in accordance with the present disclosure, using similar reference numbers as FIGS. 1 to 8 to show similar elements. For simplicity, all of the elements identified for the packaging container 10 in FIGS. 1 to 8 will not be repeated for the packaging container 310. As with the packaging container 10, the packaging container 310 can be used to ship a medical device 100 and then also to assist in a method of using the medical device 100, as described above. In an embodiment, an example embodiment of a medical package includes the packaging container 310 and the medical device 100.

As seen in FIGS. 17 to 20, the packaging container includes an outer part 312 and an inner part 314. The outer part 312 includes an upper portion 316 and a lower portion 318. The outer part 312 also includes a tear line 320 that separates the upper portion 316 and the lower portion 318. The outer part 312 further includes a punch out section 321 located adjacent to the tear line 320, such that a user can place a finger in the punch out section 321 to grip and tear the upper portion 316 off of the lower portion 318 along the tear line 320. The lower portion 318 contains the inner part 314 when the upper portion 316 is removed using the tear line 320.

The inner part 314 is configured to hold one medical device 100 both during shipping and upright during use of the medical device 100, while the outer part 312 is configured to form an enclosure for the inner part 314 which can be separated to expose the inner part 314 during use of the medical device 100. Like above, the inner part 314 can formed by a first single sheet of cardboard folded along a plurality of fold lines to form at least one rack surface 366, 368 configured to hold a medical device 100 both during shipping and upright during use of the medical device 100, and the outer part 312 can be formed by a second single sheet of cardboard folded along a plurality of fold lines to form an enclosure for the inner part 314.

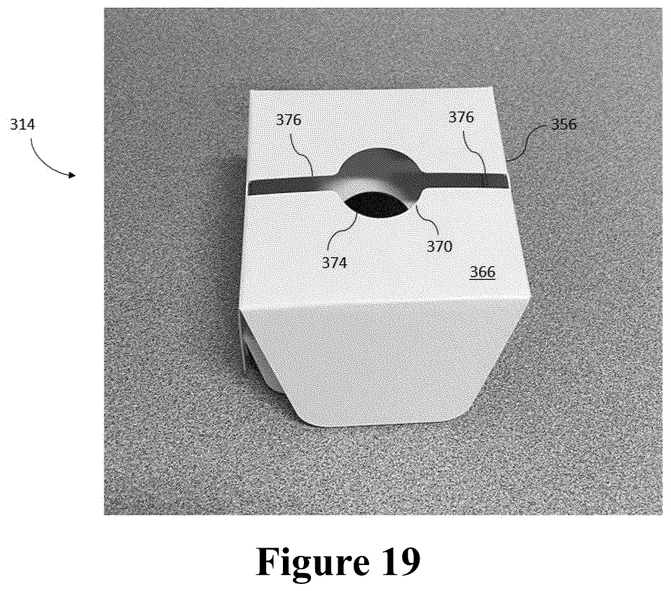

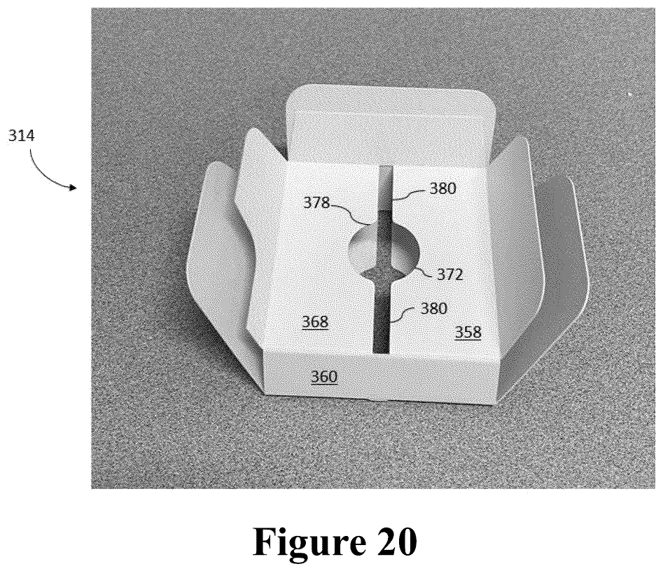

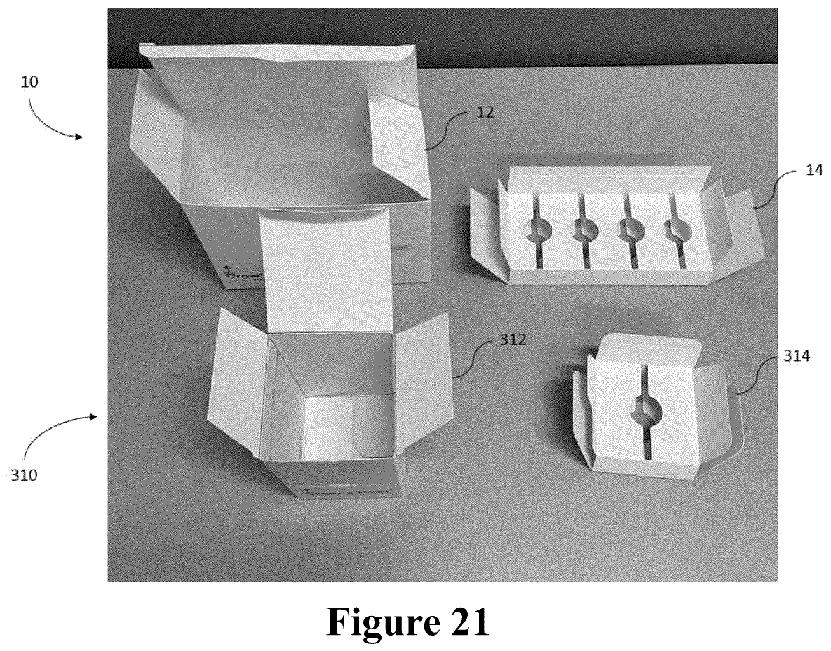

FIGS. 19 and 20 show the inner part 314 before the inner part 314 is combined with the outer part 312. Similar to the embodiment of FIGS. 1 to 8, the inner part 314 includes an upper portion 356 and a lower portion 358. The inner part 314 also includes an intermediate portion 360 located between the upper portion 356 and the lower portion 358. The upper portion 356 is connected to the intermediate portion 360 by a fold line, and the lower portion 358 is connected to the intermediate portion 360 by a fold line. The inner part 314 includes an upper rack surface 366 and a lower rack surface 368. More specifically, the upper portion 356 includes the upper rack surface 366, and the lower portion 358 includes the lower rack surface 368. The upper rack surface 366 includes an upper cutout 370. The upper cutout 370 is configured to hold a medical device 100 both during shipping and upright during use of the medical device 100. Similarly, the lower rack surface 368 includes a lower cutout 372. The lower cutout 372 is also configured to hold a medical device 100 both during shipping and upright during use of the medical device 100. The lower cutout 372 aligns with the upper cutout 370 in the vertical direction.

Similar to the embodiment of FIGS. 1 to 8, the upper cutout 370 includes a center portion 374 and two side portions 376. The side portions 376 form an aperture in combination with the center portion 374. Likewise, the lower cutout 372 includes a center portion 378 and two side portions 380. The side portions 380 form an aperture in combination with the center portion 378. The center portions 374, 378 are circular, and the side portions 376, 380 are rectangular and extend from opposite sides of the center portions 74, 78 to opposing fold lines. Those of ordinary skill in the art will recognize from this disclosure that the upper cutouts 370 and the lower cutouts 372 can be made in different sizes, shapes or dimensions and/or be placed at different locations.

FIG. 21 illustrates the outer part 12 and the inner part 14 of the packaging container 10, and the outer part 312 and the inner part 314 of the packaging container packaging container 310. The packaging container 10 is created by constructing and inserting the inner part 14 into the outer part 12, for example as described herein. The packaging container 310 is created by constructing and inserting the inner part 314 into the outer part 312, for example as described herein. In an embodiment, medical devices 100 can be packaged in both the container 10 and the packaging container 310 and selectively sent to hospitals or clinical labs based on the number of medical devices 100 needed.

It should be understood that various changes and modifications to the apparatuses and methods described herein will be apparent to those skilled in the art and can be made without diminishing the intended advantages.

### GENERAL INTERPRETATION OF TERMS

In understanding the scope of the present invention, the term “comprising” and its derivatives, as used herein, are intended to be open-ended terms that specify the presence of the stated features, elements, components, groups, and/or steps, but do not exclude the presence of other unstated features, elements, components, groups, integers and/or steps. The foregoing also applies to words having similar meanings such as the terms, “including”, “having” and their derivatives. Also, the terms “part,” “section,” or “element” when used in the singular can have the dual meaning of a single part or a plurality of parts.

The term “configured” as used herein to describe a component, section or part of a device includes hardware that is constructed to carry out the desired function.

While only selected embodiments have been chosen to illustrate the present invention, it will be apparent to those skilled in the art from this disclosure that various changes and modifications can be made herein without departing from the scope of the invention as defined in the appended claims. For example, the size, shape, location or orientation of the various components can be changed as needed and/or desired. Components that are shown directly connected or contacting each other can have intermediate structures disposed between them. The functions of one element can be performed by two, and vice versa. The structures and functions of one embodiment can be adopted in another embodiment. It is not necessary for all advantages to be present in a particular embodiment at the same time. Every feature which is unique from the prior art, alone or in combination with other features, also should be considered a separate description of further inventions by the applicant, including the structural and/or functional concepts embodied by such features. Thus, the foregoing descriptions of the embodiments according to the present invention are provided for illustration only, and not for the purpose of limiting the invention as defined by the appended claims and their equivalents.

What is claimed is:

1. A packaging container for one or more medical device, the packaging container comprising:
an inner part including a rack surface having at least one cutout configured to hold the one or more medical device both during shipping and upright during use of the one or more medical device; and
an outer part enclosing the inner part, the outer part including a tear line enabling a user to remove a portion thereof and expose the rack surface of the inner part so that the user can use the one or more medical device while the one or more medical device is held upright by the inner part.

2. The packaging container of claim 1, wherein
the inner part and the outer part are each formed by a single sheet of cardboard.

3. The packaging container of claim 1, wherein
the at least one cutout is formed as an aperture in the rack surface.

4. The packaging container of claim 1, wherein
the at least one cutout includes a center portion and at least one side portion, the at least one side portion forming an aperture in combination with the center portion, the at least one side portion extending outwardly from the center portion in a first direction and being smaller than the center portion in a second direction perpendicular to the first direction.

5. The packaging container of claim 1, wherein
the outer part includes an upper portion and a lower portion,
the upper portion is configured to be separated from the lower portion using the tear line, and
the lower portion contains the inner part when the upper portion is removed using the tear line.

6. The packaging container of claim 1, wherein
the inner part includes an upper rack surface and a lower rack surface,
the upper rack surface includes at least one upper cutout configured to hold the one or more medical device upright during use of the one or more medical device, and
the lower rack surface includes at least one lower cutout aligned with the upper cutout in a vertical direction, the at least one lower cutout configured to hold the one or more medical device upright during use of the one or more medical device.

7. The packaging container of claim 1, wherein
the rack surface includes a plurality of cutouts configured to each hold a medical device upright both during shipping and during use of the medical device.

8. A packaging container for one or more medical device, the packaging container comprising:
an inner part formed by a first single sheet of cardboard folded along a plurality of fold lines to form at least a rack surface configured to hold the one or more medical device both during shipping and upright during use of the one or more medical device; and
an outer part formed by a second single sheet of cardboard folded along a plurality of fold lines to form an enclosure for the inner part, the outer part including an upper portion and a lower portion, the upper portion configured to be separated from the lower portion to expose the inner part.

9. The packaging container of claim 8, wherein
the first single sheet of cardboard and the second single sheet of cardboard are each part of a same single sheet of cardboard.

10. The packaging container of claim 8, wherein
the inner part includes at least one cutout configured to hold the one or more medical device upright during use of the one or more medical device.

11. The packaging container of claim 9, wherein
the at least one cutout includes a center portion and at least one side portion, the at least one side portion forming an aperture in combination with the center portion, the at least one side portion extending outwardly from the center portion in a first direction and being smaller than the center portion in a second direction perpendicular to the first direction.

12. The packaging container of claim 8, wherein
the outer part includes a tear line, and
the upper portion is configured to be removed from the lower portion using the tear line.

13. The packaging container of claim 8, wherein
the inner part includes an upper rack surface and a lower rack surface,
the upper rack surface includes at least one upper cutout configured to hold the one or more medical device upright during use of the one or more medical device, and
the lower rack surface includes at least one lower cutout aligned with the upper cutout in a vertical direction, the at least one lower cutout configured to hold the one or more medical device upright during use of the one or more medical device.

14. The packaging container of claim 8, wherein
the rack surface includes a plurality of cutouts configured to each hold a medical device both during shipping and upright during use of the medical device.

15. A medical package comprising:
one or more medical device; and
a packaging container containing the one or more medical device therein, the packaging container having at least one cutout configured to hold the one or more medical device both during shipping and upright during use of the one or more medical device.

16. The medical package of claim 15, wherein
the packaging container includes an inner part and an outer part,
the inner part includes a rack surface having at least one cutout configured to hold the one or more medical device both during shipping and upright during use of the one or more medical device, and
the outer part encloses the inner part and includes a tear line enabling a user to remove a portion thereof and expose the rack surface of the inner part so that the user can use the one or more medical device while the one or more medical device is held upright by the inner part.

17. The medical package of claim 15, wherein
the packaging container includes an inner part and an outer part,
the inner part is formed by a first single sheet of cardboard folded along a plurality of fold lines to form at least a rack surface configured to hold the one or more medical device both during shipping and upright during use of the one or more medical device, and
the outer part is formed by a second single sheet of cardboard folded along a plurality of fold lines to form an enclosure for the inner part, the outer part including an upper portion and a lower portion, the upper portion configured to be separated from the lower portion to expose the inner part.

18. The medical package of claim 15, wherein
the one or more medical device separates a tissue sample from dislodged cells obtained during a biopsy.

19. The medical package of claim 15, wherein
the one or more medical device includes a buffer container and a sample collection container, the buffer container removeable from the sample collection container while the sample collection container is help upright within the at least one cutout.

20. The medical package of claim 15, wherein
the one or more medical device includes a plurality of medical devices that are each individually sealed in individual device packaging,
the at least one cutout includes a device portion and at least one side portion, and
the plurality of medical devices that are each individually sealed are each positioned within a respective cutout with the medical device located in the device portion and the packaging located in the side portion.
